(19) United States
(12) Patent Application Publication
(10) Pub. No.: US 2006/0018043 A1
(43) Pub. Date: Jan. 26, 2006

(54) Apparatus and Method for Testing Ovulation/Fertility Utilizing Saliva

(76) Inventor: Gerard Gontier, Las Vegas, NV (US)

Correspondence Address: JOHN C. SMITH, ESQ., 2499 GLADES ROAD, SUITE 113, BOCA RATON, FL, 33431, US

(21) Appl. No.: 11/161,164

(22) Filed: Jul. 25, 2005

Related U.S. Application Data
(60) Provisional application No. 60/521,944, filed on Jul. 25, 2004.

(51) Int. Cl. G02B 27/02; A61B 10/00

(52) U.S. Cl. 359/804; 600/551

(57) ABSTRACT

A portable handheld fertility/ovulation tester that uses ambient light, and requires no power. A unique sample holding frame and adjustable lens assembly is inserted into a light chamber in the bottom of the tester. An aperture in the bottom of the chamber is aligned with a microscope lens assembly and is sized to provide the proper amount of ambient light for the microscope lens assembly. The light entering the chamber and its direction are controlled by the size and shape of the aperture, which may also have an optional light gathering lens to increase illumination. The adjustable lens assembly is threaded into a sample plate frame having a transparent sample plate. The microscope lens is aligned with the transparent sample plate and the aperture such that light from the aperture which strikes the bottom of the sample plate frame passes through the simple plate and enters the microscope lens where it is magnified for viewing. The microscope lens assembly is removably mounted onto the light chamber. The lens assembly is mounted onto the light chamber such that when the fertility ovulation tester is held with the aperture pointed towards a light source, a ferning pattern in the saliva sample placed on the transparent sample plate can be easily viewed through the microscope lens. The device also uses an external cover to secure the components of the fertility ovulation tester when not in use.

### CROSS-REFERENCE TO RELATED APPLICATIONS

This application is related to, and claims the benefit of, the provisional patent application entitled “Apparatus and Method for Testing Ovulation/Fertility Utilizing Saliva”, filed Jul. 25, 2004, bearing U.S. Ser. No. 60/521,944 and naming Gerard Gontier, the named inventor herein, as sole inventor, the contents of which is specifically incorporated by reference herein in its entirety.

### BACKGROUND OF THE INVENTION

1. Technical Field

The present invention relates to fertility testing devices. In particular, it relates to portable devices, illuminated by ambient light, which are capable of detecting when a female is ovulating or fertile.

2. Background

Many women have difficulty becoming pregnant for a variety of reasons. One common problem is associated with timing. Due to the normal ovulation process, a healthy female typically has only a few days each month in which she can become pregnant. Usually, there is a period of four or five days during the menstrual cycle when an attempt at pregnancy is most likely to succeed. For most women, this is approximately 14 days after the beginning of menstruation in a 28 day cycle. If she is inseminated outside of this small time window, she will not become pregnant. As a result, when a female is seeking to become pregnant it is important to identify when the fertility/ovulation time window has opened, so that insemination can be performed with successful results.

It is known that the ovulation cycle produces a side effect related to saliva. In particular, when a female is in ovulation, her body produces increased amounts of estrogen which causes an egg to be released. A side effect of this elevated estrogen process is an increased saliva saline level. When the saliva saline level (i.e., the relative amount of salt in saliva) is increased, an observable change can be seen in the saliva when it is dried. In fact, during the fertility/ovulation time window, the saline level of saliva is increased to the point where saliva produces a distinct crystalline structure when it dries.

The crystalline structure formed by the saliva resembles a fern-like pattern which has a unique visual appearance. This crystalline structure is substantially different from that produced by saliva with lower salt levels, and is easily recognizable under magnification. The creation of this fern-like crystalline structure is typically referred to as “ferning.” As a result of this estrogen caused side effect, examining the dried saliva of a female for ferning can provide a good indication of whether or not the fertility/ovulation window is open. Further, peak times within the ovulation window can be determined by how clear and well formed the ferning structure is.

Prior attempts to take advantage of this phenomenon have involved the use of complicated and expensive microscope devices which are either cumbersome to work with, overly complicated due to unnecessary mechanical or electronic components. Prior art devices typically require battery-powered illumination systems which use a variety of lamp technologies, such as incandescent lamps, LEDs, etc. These additional components add to the cost of manufacturing device, increase the expense of operation, and increase the likelihood of failure. In addition, these devices are very inconvenient for a woman to carry with her for the purpose of periodically testing her state of fertility. It would be desirable to have a simple structure with a minimal number of components, which does not require an internal electrical power source, which could take advantage of ambient light for testing saliva for a ferning pattern, and which a woman could conveniently carry in a purse.

### SUMMARY OF THE INVENTION

The present invention provides a portable handheld fertility/ovulation tester which works on ambient light, and requires no power. It has a compact size which allows it to be conveniently carried in a pocket or purse. It uses a manually adjustable microscope lens assembly which has a transparent sample plate integrated into the bottom of a sample plate frame. The lens assembly also has an adjustable microscope lens that is threaded into the sample plate frame and is aligned with the transparent sample plate such that light striking the bottom of the sample plate frame passes through the simple plate and enters the microscope lens where it is magnified for viewing. The microscope lens assembly is removably mounted onto a light chamber. The light chamber has an aperture in the bottom which allows ambient light to enter the light chamber and be directed towards the sample plate. The aperture is sized to control light entering the light chamber such that both the amount of light entering the chamber and the direction of the light are controlled by the size and shape of the aperture. The lens assembly is mounted onto the light chamber such that when the fertility ovulation tester is held with the aperture pointed towards a light source, a saliva sample placed on the transparent sample plate can be easily viewed through the microscope lens. The device also uses an external cover to secure the components of the fertility ovulation tester when not in use.

### BRIEF DESCRIPTION OF THE DRAWINGS

FIG. 1 is a side external view of the fertility/ovulation detection device with the microscope cover secured to the light chamber.

FIG. 2 is a side external view of the fertility/ovulation detection device with the microscope cover detached from the light chamber.

FIG. 3 is a side cutaway view of the fertility/ovulation detection device with the microscope cover detached from the light chamber.

FIGS. 4A-B illustrate the method of use of the fertility/ovulation detection device.

FIG. 5A illustrates a bottom view of the microscope assembly which shows the location of the light aperture.

FIG. 5A illustrates a top view of microscope assembly that shows the location of the aperture into which the lens and slide assembly is inserted.

FIG. 6 illustrates an alternative preferred embodiment which uses a lens assembly to increase the amount of light entering the light chamber and to the slide.

FIG. 7 illustrates the patterns created by dry saliva dependent on the effects of estrogen and the menstrual cycle.

### DESCRIPTION OF THE PREFERRED EMBODIMENT

Prior to a detailed discussion of the figures, a general overview of the system will be presented. The invention takes advantage of a phenomenon caused by the female hormone system. During the normal menstrual cycle, estrogen levels in the female body vary widely. During most of the menstrual cycle, estrogen levels are low, but when producing an egg for reproduction, the body produces elevated levels of estrogen.

A side effect of these elevated estrogen levels is a change in the chemical composition of saliva that produces distinct changes in the microscopic appearance of the saliva when it dries. At low estrogen levels, saliva dries in a distinct amorphous pattern when viewed under a microscope. Due to the rising estrogen levels which occur when ovulation approaches, and the saliva begins to form a crystalline structure, rather than and amorphous pattern. During the peak levels of estrogen, the period of fertility, this crystalline structure forms a unique ferning pattern when it dries. This firming pattern is distinctly visible under a microscope. As a result, by detecting the ferning pattern, women now have the advantage of being able to use a simple noninvasive test, which they can self-administer, that allows them to monitor their fertility on a daily basis to determine when they are most likely to become pregnant.

This invention takes advantage of this phenomenon by providing a handheld fertility/ovulation testing microscope which uses ambient light for illumination. The device has a microscope assembly which includes a detachable lens and slide assembly which is used in combination with a light chamber. The lens and slide assembly further includes an adjustable focus mechanism and a magnification lens that is aligned with a slide for mounting a saliva sample. The lens and slide assembly is removably attached to a microscope assembly which includes a light chamber designed to direct ambient light to the lens and slide assembly. In one alternative embodiment, the light chamber further includes an optional light gathering lens which is used to increase the amount of light entering the light chamber. When not in use, a detachable microscope cover is attached to the microscope assembly to protect the lens and slide assembly from damage.

When fertility/ovulation testing is desired, the microscope cover is removed from the microscope assembly. Then the lens and slide assembly is detached from the microscope assembly to expose the slide which is on the bottom of the lens and slide assembly. A small sample of saliva is placed on the slide and allowed to dry. This usually takes a few minutes (i.e. 2-5 minutes). Once the saliva is dry, the lens and slide assembly is reattached to the microscope assembly. The microscope assembly also includes an aperture for directing light to the slide assembly to illuminate the saliva sample. The aperture, the slide, and the lens are arranged such that light entering the aperture travels through the light chamber inside microscope assembly, passes through the saliva on the slide, and travels through the lens where it is magnified and made available for the user to view. The lens is mounted such that it can be adjustably focused on the dried saliva sample to give the best possible image.

To view the image, the user holds a microscope assembly to an ambient light source while looking through the lens. The ambient light illuminates the sample, and the user adjusts the lens for the sharpest and best focused image.

The use of ambient light provides the advantages over electronic devices in that the user does not have to depend on batteries, illumination devices, switches or other electronic components which will fail over time. The ambient light system provides a device that does not rely on a variety of components to work, and is therefore more reliable than electronic devices. Further, because it provides a structure with a minimum number of components, it is both reliable and inexpensive to manufacture.

Alternative embodiments include methods of increasing the amount of ambient light made available to view the saliva sample. For example, an optional light gathering lens can be placed in the aperture (which may be expanded to allow use of a larger lens) to increase the amount of ambient light directed at the sample slide.

Having discussed the fertility/ovulation tester in general, we turn now to a more detailed discussion of the figures.

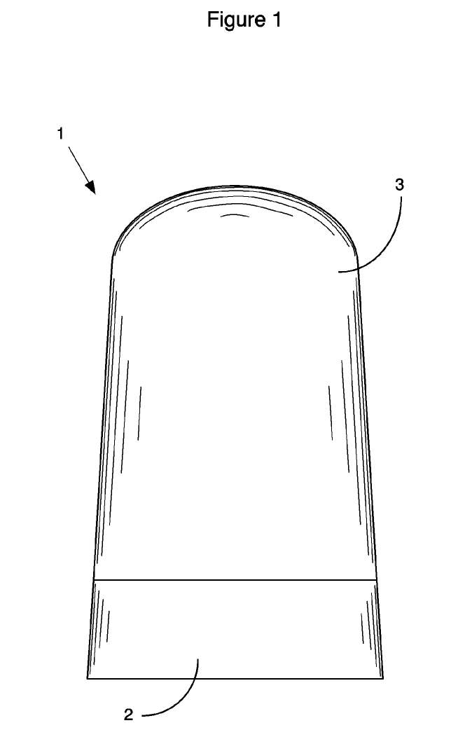

FIG. 1 illustrates a side external view of a preferred embodiment of the fertility/ovulation detection device 1 with the microscope cover 3 secured to the microscope assembly 2. Microscope cover 3 and the microscope assembly 2 can be fabricated from any suitable material. In the preferred embodiment, it is fabricated from plastic to provide low weight, resistance to water and other environmental factors, and resistance to impact damage. The device is also shaped such that it has a substantially flattened configuration for ease of handling and storing.

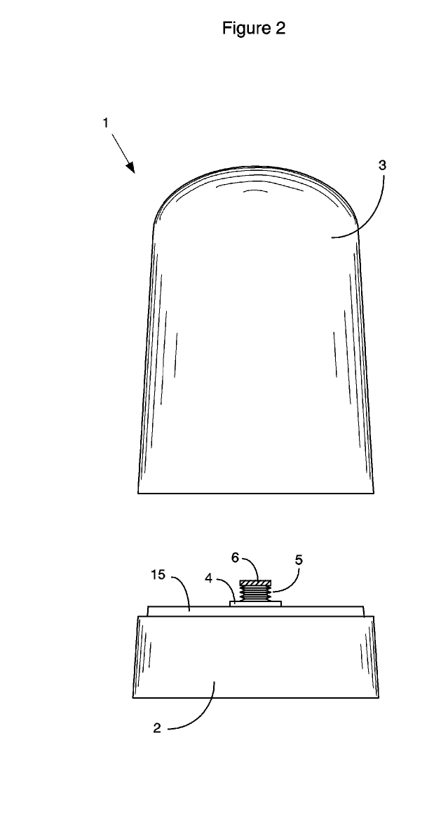

In FIG. 2, the microscope cover 3 is shown removed from the microscope assembly 2. In the preferred embodiment, the microscope cover 3 is secured to the microscope assembly 2 by a ridge 15 which uses frictional pressure to hold the microscope cover 3 place when not in use.

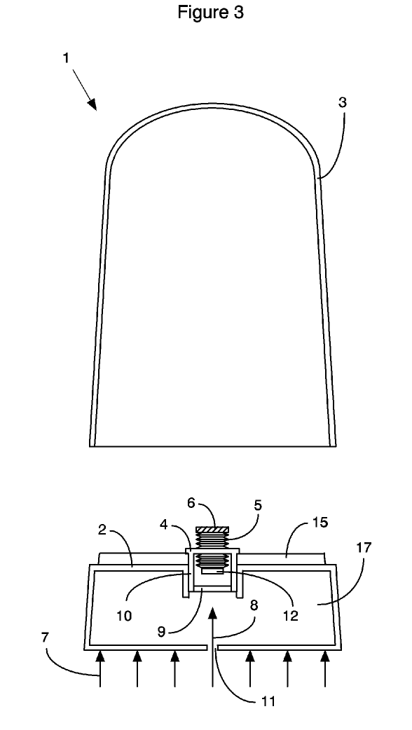

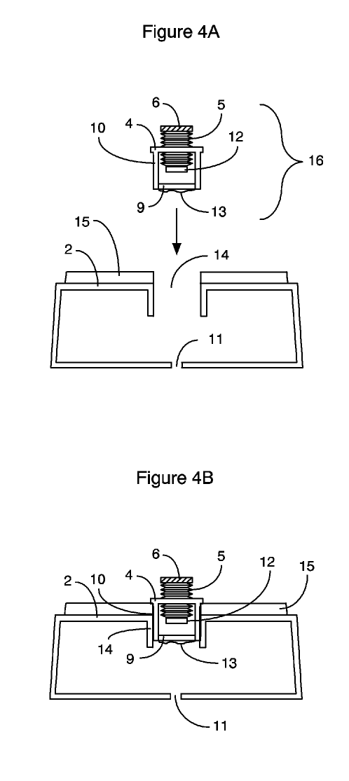

A side view of the microscope assembly 2 is shown in which the upper portion of the lens and slide assembly 16 a shown extending above the upper surface of the microscope assembly. This upper portion is comprised of a knurled edge 6 which is attached to the upper end of threaded shaft 5. The threaded shaft 5 is in turn threadably attached to lens support 4. When using the device to monitor fertility/ovulation, the user will rotate the knurled edge 6 to adjust the focus of the lens 12 (illustrated below in FIGS. 3 and 4A-B). The interior channel of the lens support 4 is also threaded to accommodate the threaded shaft 5 such that the lens can be focused when the knurled edge 6 is rotated.

FIG. 3 shows a cutaway side view of the preferred embodiment of FIG. 2. In this embodiment, an aperture 11 allows ambient light 7 to enter the internal light chamber 17 of the microscope assembly 2. In addition to aperture 11, the lens and slide assembly 16 is also shown mounted through an aperture in the top surface of the microscope assembly 2. The lens and slide assembly 16 includes the knurled edge 6, the threaded shaft 5, the lens support 4, the slide 9, and the lens 12. The lens support 4 also includes vertical extensions 10 which support and secure the lens support 4 to the microscope assembly 2.

The aperture 11 and the slide 9 are aligned with one another such that light entering the aperture 11 will pass through the slide 9 and enter the lens 12 where the image will be magnified and directed upward through an aperture in the knurled edge 6 to the eye of the user.

An advantage provided by the light chamber 17 is that it controls the light striking the slide 9. Further, it controls the direction that light strikes slide 9 surface. This avoids the situation where light striking slide 9 surface may actually interfere with proper viewing of the dried saliva due to refraction or other problems. The light chamber 17 allows the user to select an ambient light 17 source and prevents other light sources from interfering with the illumination of the slide 9.

FIGS. 4A-B illustrates the method of use of the fertility/ovulation detection device 1. In FIG. 4A, the lens and slide assembly 16 is removed from the microscope assembly 2. In the preferred embodiment, the lens and slide assembly 16 secured to the microscope assembly 2 via a slidable pressure fit. However those skilled in the art will recognize that any suitable method can be used to secure the lens and slide assembly 16. For example, a threaded engagement can be used to attach lens and slide assembly 16 to the microscope assembly 2.

Once the lens and slide assembly 16 is removed from the microscope assembly 2, the user will place a layer of saliva 13 onto the slide 9. The saliva will be allowed to dry prior to reattaching the lens and slide assembly 16 to the microscope assembly 2. This usually takes approximately two to five minutes. During this time, the ferning process will take place if the woman is fertile or ovulating. If the woman is not fertile or ovulating, than the ferning process will not take place.

In FIG. 4B, the next step in the process is illustrated. A lens and slide assembly 16 is reinserted into the microscope assembly 2. At this point, the lens 12, the slide 9, and the aperture 11 are now in alignment such that light entering aperture 11 illuminates the dried saliva 13 on slide 9. Once the lens and slide assembly 16 is reattached to the microscope assembly 2, the user looks through the lens and slide assembly 16 while holding it such that aperture 11 is pointed toward a light source. At the same time, the user adjusts the focus by rotating the knurled edge 6.

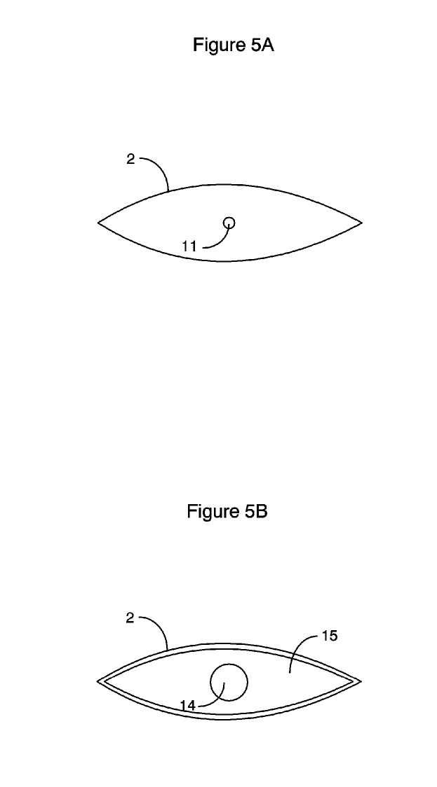

FIG. 5A illustrates a bottom view of the microscope assembly 2 which shows the location of the light aperture 11. In this illustration, the light aperture 11 is simply an open aperture. However, those skilled in the art will recognize that the aperture can also be sealed with a transparent material that will allow light 7 to penetrate the light chamber 17. The advantage associated with the transparent window is that it prevents dirt or moisture from entering the light chamber 17.

FIG. 5B illustrates a top view of the microscope assembly 2 that shows the location of the aperture 14 into which the lens and slide assembly 16 is inserted. Also illustrated in FIGS. 5A-B is the flattened shape of the fertility/ovulation detection device 1. This allows the fertility/ovulation detection device 1 to be conveniently stored when not in use, and also provides a structure which is easy to grasp and hold.

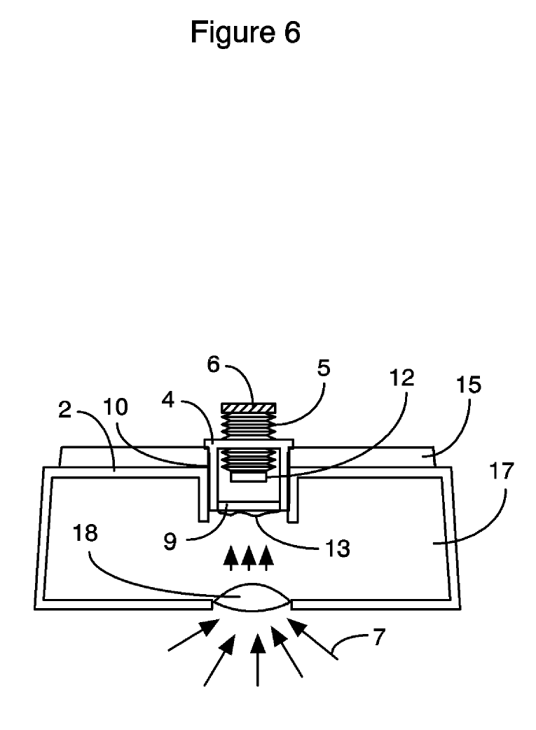

FIG. 6 illustrates an alternative preferred embodiment which uses a light gathering lens 18 to increase the amount of light entering the light chamber 17. Light gathering lens 18 also focuses the light on the slide 9 to increase the amount of light available to the user when viewing the slide 9.

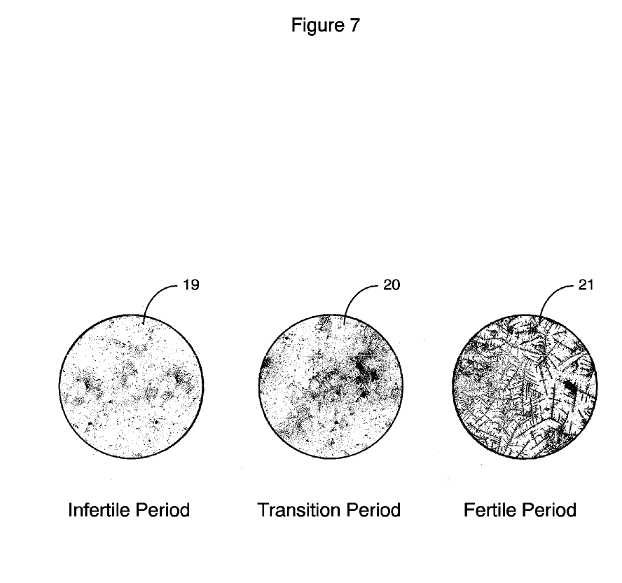

FIG. 7 illustrates the patterns created by dry saliva dependent on the effects of estrogen and the menstrual cycle. In this figure, view 19 illustrates the amorphous pattern created in the infertile period when a female is not fertile or ovulating. View 20 illustrates the transition period when the ferning pattern is just beginning to form and the female is approaching ovulation. View 21 illustrates the ferning pattern produced by dried saliva during the period when a female is ovulating and fertile.

As can be seen from the foregoing, the invention provides a unique viewing structure for determining fertility during the normal monthly menstrual cycle. A unique removable lens structure is provided which allows for ones to be automatically aligned with the sample slide. Further, the removable lens and sample slide structures allow the lands and sample slide to be easily inserted into the light chamber and automatically aligned with the source of light. Finally, an optional light gathering lens is provided to increase light in a new light chamber. As a result of cooperation between the elements of this invention, a woman can conveniently carry and use a lightweight, and inexpensive device to determine fertility levels. It eliminates the need for the complex and expensive devices which heretofore were inconvenient for a woman to carry.

While the invention has been described with respect to a preferred embodiment thereof, it will be understood by those skilled in the art that various changes in detail may be made therein without departing from the spirit, scope, and teaching of the invention. For example, the material used to construct the fertility/ovulation tester may be anything suitable for its purpose, the size and shape of the fertility/ovulation tester can vary. The type of lens can vary, etc. Accordingly, the invention herein disclosed is to be limited only as specified in the following claims.

I claim:

What is claimed is:

1. A fertility/ovulation testing device, further comprising:
a microscope lens;
a sample slide having means to align with the microscope lens; and
ambient light control means to control the input of ambient light from a preselected ambient light source such that the ambient light passes through the sample slide and into the microscope lens.

2. A testing device, as in claim 1, further comprising:
a slide frame secured to the sample slide, and further having means to removably attach to the ambient light control means.

3. A testing device, as in claim 2, further comprising:
means to removably attach the microscope lens to the slide frame such that it is aligned with the sample slide.

4. A testing device, as in claim 3, further comprising:
means to rotate attach the microscope lens in relation to the sample slide such that the microscope lens can focus on the slide frame.

5. A testing device, as in claim 3, wherein the ambient light control means further comprises:
a light gathering lens, attached to the ambient light control means and positions such that light entering the light gathering lens is directed through the sample slide and into the microscope lens.

6. A testing device, as in claim 2, wherein the ambient light control means further comprises:
a light chamber, further comprising:
a substantially opaque wall preventing random ambient light from entering the like chamber; and
an aperture in the light chamber to allow light from a preselected light source to enter the light chamber, the aperture, when the light chamber is secured to the slide frame, aligns with the sample slide and with the microscope lens such that the sample slide is illuminated for viewing by the microscope lens.

7. A testing device, as in claim 6, further comprising:
a slide frame secured to the sample slide, and further having means to removably attach to the ambient light control means.

8. A testing device, as in claim 7, further comprising:
means to removably attach the microscope lens to the slide frame such that it is aligned with the sample slide.

9. A testing device, as in claim 8, further comprising:
means to rotate attach the microscope lens in relation to the sample slide such that the microscope lens can focus on the slide frame.

10. A testing device, as in claim 9, wherein the ambient light control means further comprises:
a light gathering lens, attached to the ambient light control means and positions such that light entering the light gathering lens is directed through the sample slide and into the microscope lens.

11. A fertility/ovulation testing device, further comprising:
a microscope lens assembly, the microscope lens assembly having means to secure to the microscope lens;
a sample slide having means removably attach to the microscope lens assembly, such that the sample slide aligns with the microscope lens; and
a light chamber having a substantially opaque wall preventing random ambient light from entering the light chamber, and an aperture in the light chamber to allow light from a preselected light source to enter the light chamber.

12. A testing device, as in claim 11, further comprising:
a slide frame secured to the sample slide, and further having means to removably attach to the ambient light control means.

13. A testing device, as in claim 12, further comprising:
means to removably attach the microscope lens to the slide frame such that it is aligned with the sample slide.

14. A testing device, as in claim 13, further comprising:
means to rotate attach the microscope lens in relation to the sample slide such that the microscope lens can focus on the slide frame.

15. A testing device, as in claim 13, wherein the ambient light control means further comprises:
a light gathering lens, attached to the ambient light control means and positions such that light entering the light gathering lens is directed through the sample slide and into the microscope lens.

16. A method of determining when a woman is fertile, including the steps of:
drying a saliva sample;
examining the dried saliva sample to determine if a ferning pattern is present; and
illuminating the dried saliva sample with ambient light that comes from a preselected ambient light source, and preventing ambient light from other sources from illuminating the dried saliva samples;
whereby, the woman is determined to be fertile if a burning pattern is present, and infertile if the ferning pattern is absent.

17. A method, as in claim 16, including the additional step of:
controlling the ambient light source which illuminates the dry saliva sample by enclosing the saliva sample in an opaque light chamber and only permitting ambient light to enter the light chamber through an aperture.

18. A method, as in claim 1 7, including the additional steps of:
using a simple slide to dry the saliva sample; and
using an adjustable microscope lens to view the saliva sample.

19. A method, as in claim 1 8, including the additional steps of:
gathering ambient light from a wide range of what sources; and
focusing the gathered ambient light and using it to illuminate the saliva samples.

20. A method, as in claim 19, including the additional step of:
using a light gathering lens to gather the ambient light.
